(19) United States
(12) Patent Application Publication
(10) Pub. No.: US 2017/0325477 A1
(43) Pub. Date: Nov. 16, 2017

(54) Yogurt Premix for Canines

(71) Applicant: Roger Allen Glade, Agoura Hills, CA (US)

(72) Inventor: Roger Allen Glade, Agoura Hills, CA (US)

(21) Appl. No.: 15/595,520

(22) Filed: May 15, 2017

Related U.S. Application Data
(60) Provisional application No. 62/335,994, filed on May 13, 2016.

(51) Int. Cl. A23G 9/04; A23K 50/40; A23K 10/18; A23K 20/174; A61K 35/20; A23K 50/00; A61K 9/00

(52) U.S. Cl. CPC A23G 9/04; A23K 10/18; A23K 50/40; A23K 20/174; A23K 50/00; A61K 9/0087; A61K 35/20

(57) ABSTRACT

A yogurt premix for canine provides both a flavorful and nutritional treat for canines. The yogurt premix includes a plurality of ingredients that each has their own unique beneficial properties. The plurality of ingredients includes a quantity of powdered yogurt, a quantity of protein powder, a quantity of thickening powder, a quantity of dried apple, a quantity of powdered banana, and a quantity of coconut oil. The quantity of powdered yogurt, the quantity of protein powder, the quantity of thickening powder, the quantity of dried apple, the quantity of powdered banana, and the quantity of coconut oil are heterogeneously mixed into the quantity of powdered yogurt. At least one flavor additive provides specific flavors to the yogurt premix that accommodates a canine's preferences.

The current application claims a priority to the U.S. Provisional Patent application Ser. No. 62/335,994 filed on May 13, 2016.

### FIELD OF THE INVENTION

The present invention relates generally to yogurt premix. More specifically, the present invention is a yogurt premix that is fabricated and flavored for canine composition.

### BACKGROUND OF THE INVENTION

Dogs love frozen treats as licking is a natural action for pets. Unfortunately, such frozen treats as ice cream and frozen yogurt contain ingredients that are not good for dogs. These harmful ingredients include milk, cream, sugar, corn syrup, artificial sweetener, artificial colors, preservatives, and so on.

The present invention serves to provide a healthy and edible treat for dogs. The present invention is a custom blend of ingredients specially selected for dogs. Yogurt provides many health benefits to dogs, and because the present invention is a powder mix, the present invention delivers active probiotics. No sugar, corn syrup, or artificial sweeteners are included in the present invention. The present invention however is enjoyable by dogs as sweetness is included by adding natural ingredients such as powdered fruits and vegetables, thereby reducing the tartness and providing extra fiber. The present invention also provides increased servings of protein. The present invention is processed in a traditional soft serve machine.

The present invention also facilitates the distribution process as well. As the present invention is a blend of powdered ingredients, the present invention may be blended and bagged without having to process the premix before distribution. Therefore, the present invention has a high shelf life and does not require special storage conditions.

The present invention also allows retailers to attract and tend to more customers. More specifically, the present invention attracts customers with dogs and allows customers who have dogs to treat their dogs without feeling guilty for sharing food that may not be good for them.

### BRIEF DESCRIPTION OF THE DRAWINGS

FIG. 1 is a block diagram depicting the ingredients of the preferred embodiment of the present invention.

FIG. 2 is a block diagram depicting the ratio of the ingredients within the preferred embodiment of the present invention.

FIG. 3 is a block diagram depicting the health benefits of powdered yogurt.

FIG. 4 is a block diagram depicting the ingredients of an exemplary embodiment of the present invention.

FIG. 5 is a block diagram depicting the ratio of the ingredients within an exemplary embodiment of the present invention.

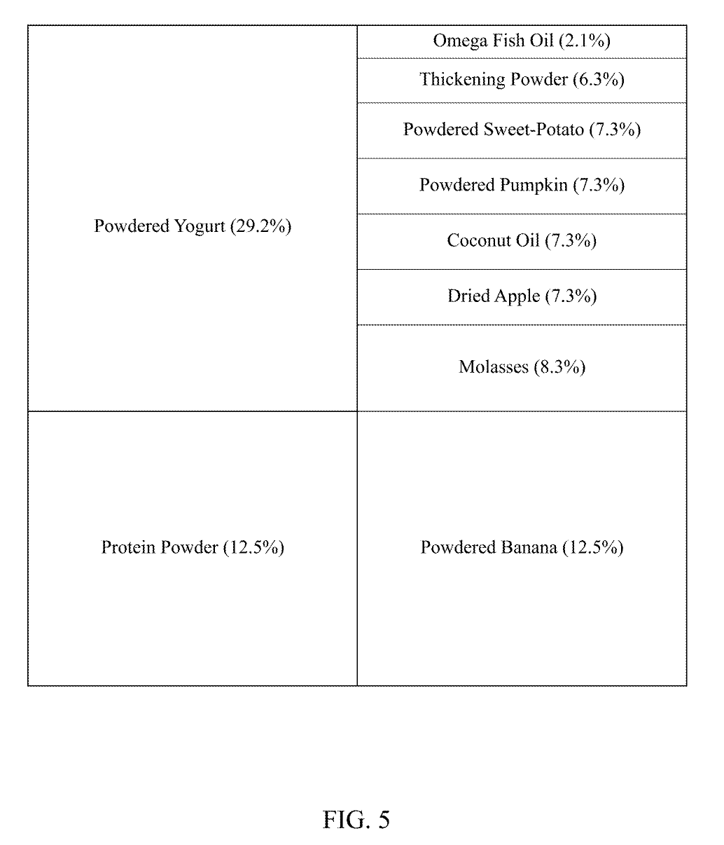

### DETAILED DESCRIPTIONS OF THE INVENTION

All illustrations of the drawings are for the purpose of describing selected versions of the present invention and are not intended to limit the scope of the present invention.

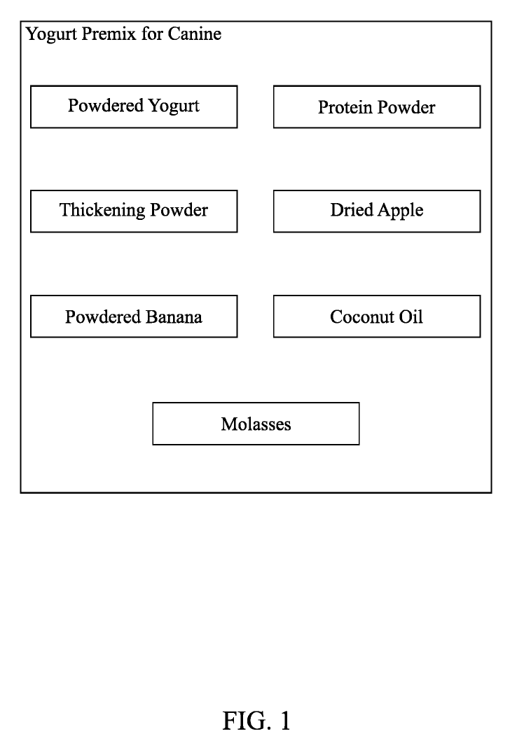

The present invention is a yogurt premix for canines that serves as a tasty and nutritional treat for dogs. The present invention offers a variety of flavors that accommodate a dog's preference. As can be seen in FIG. 1, the present invention comprises a quantity of powdered yogurt, a quantity of protein powder, a quantity of thickening powder, a quantity of dried apple, a quantity of powdered banana, a quantity of coconut oil, and a quantity of molasses. The quantity of protein powder, the quantity of thickening powder, the quantity of dried apple, the quantity of powdered banana, the quantity of coconut oil, and the quantity of molasses are heterogeneously mixed into the quantity of powdered yogurt so once the present invention is poured into the soft serve machine, a thick, evenly mixed yogurt is produced. Powdered yogurt is a powder base of the present invention and defines a custard consistency once the present invention is processed through a soft serve machine. Protein powder is a supplement that aids in the development and recovery of muscles of a dog. Protein powder enhances the overall health of the dog in addition to the body composition and performance. Thickening powder is a natural stabilizer that prevents the formation of crystals. In the preferred embodiment of the present invention, the thickening powder thickens without the use of starches. Dried apple and powdered banana both reduce the tartness of the present invention. In addition, dried apple and powdered banana provides potassium, fiber, flavonoids, and Vitamin C. Coconut oil is a natural oil extracted from coconuts which provide beneficial fats and boots the immune system response. Molasses is a natural sweetener that enhances the overall taste of the present invention without causing any stomach discomfort. The combination of these ingredients provides an edible treat for dogs that is both enjoyable and healthy.

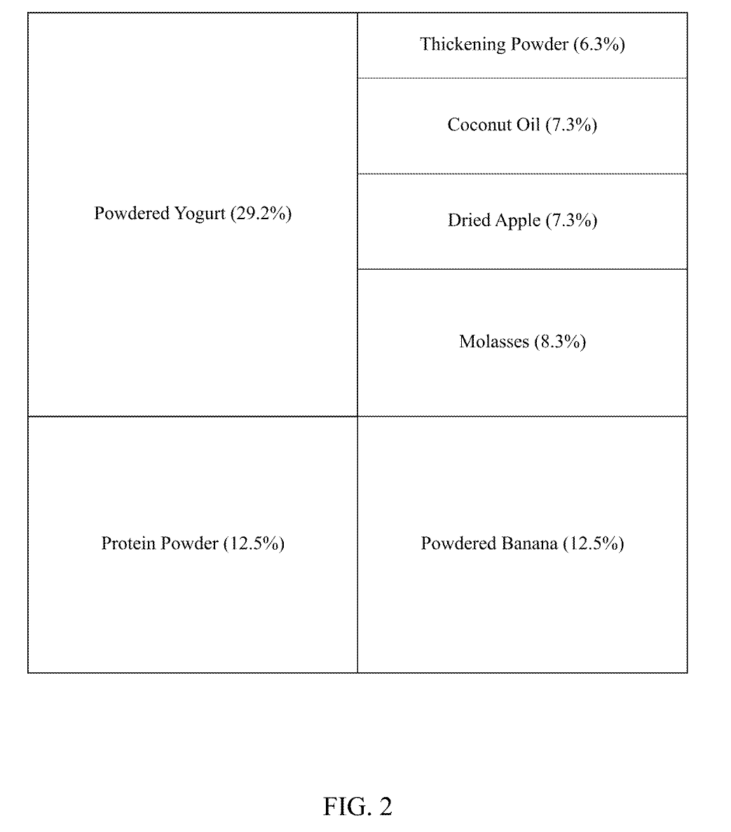

In the preferred embodiment, each of the ingredients is combined at a specific ratio within the total composition, which is shown in FIG. 2. The quantity of powdered yogurt is approximately 29.2%, percent by weight (wt %), of the yogurt premix for canines. The quantity of protein powder is approximately 12.5 wt %, of the yogurt premix for canines. The quantity of thickening powder is approximately 6.3 wt % of the yogurt premix for canines. The quantity of dried apple is approximately 7.3 wt % of the yogurt premix for canines. The quantity of powdered banana is approximately 12.5 wt % of the yogurt premix for canines. It is understood that the quantities of dried apple and powdered banana may vary based on the sweetness preference of the dog. The quantity of coconut oil is approximately 7.3 wt % of the yogurt premix for canines. The quantity of molasses is approximately 8.3 wt % of the yogurt premix for canines. The powdered yogurt is the majority of the composition as it defines the type of product and the remaining ingredients define the texture, consistency, and flavor of the composition.

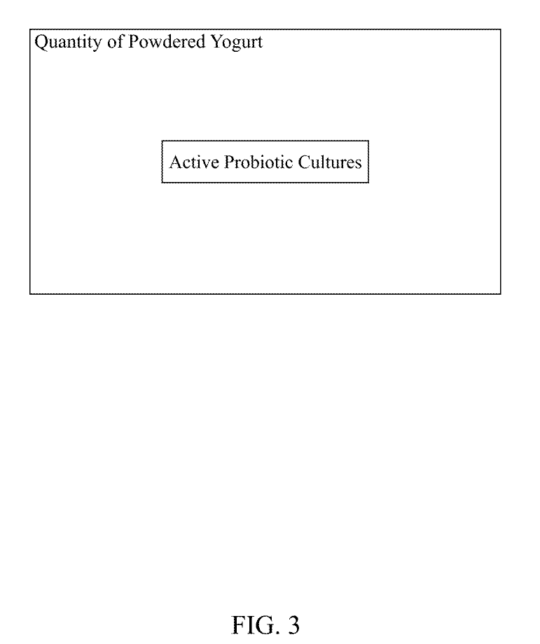

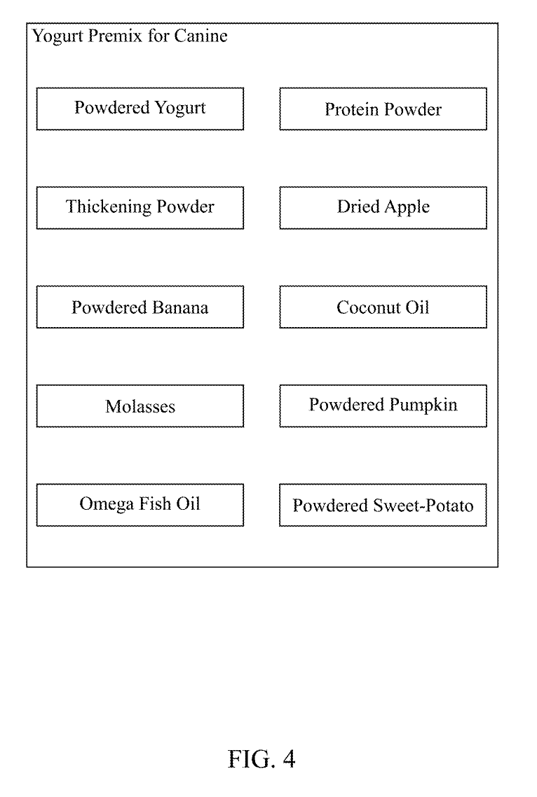

The health benefits of the yogurt premix for canines are enhanced by the preferred powdered yogurt and protein powder. The quantity of powdered yogurt includes active probiotic cultures, as illustrated in FIG. 3. Active probiotic cultures aid the digestion and modulate the immune system of dogs. The quantity of protein powder is whey protein. Whey protein rebuilds muscle and is easily digestible. Some embodiments of the present invention include a quantity of omega fish oil, as shown in FIG. 4. The quantity of omega fish oil is heterogeneously mixed into the quantity of powdered yogurt. Omega fish oil is an anti-inflammatory, reduces inflammation, and promotes a healthy coat. The quantity of omega fish oil is approximately 2.1 wt % of the yogurt premix for canines.

Some embodiments of the present invention may include a quantity of powdered pumpkin, as shown in FIG. 4. The quantity of powdered pumpkin is heterogeneously mixed into the quantity of powdered yogurt and is approximately 7.3%, by weight, of the yogurt premix for canines. This embodiment of the present invention provides a pumpkin flavored yogurt. Also, some embodiments of the present invention may include a quantity of powdered sweet-potato, also shown in FIG. 4. The quantity of sweet-potato is heterogeneously mixed into the quantity of powdered yogurt and is approximately 7.3 wt % of the yogurt premix for canines. This embodiment of the present invention provides a sweet potato flavored yogurt. Various embodiments of the present invention may comprise at least one flavor additive in order to provide a variety of flavors to a dog. The at least one flavor additive is heterogeneously mixed into the quantity of powdered yogurt. The at least one flavor additive is selected from a group consisting of: a peanut-butter flavor, a carob flavor, a pumpkin flavor, a cinnamon flavor, a sweet-potato flavor, an apple flavor, a coconut flavor, and a combination thereof. Each of the flavor additives produce a yogurt with corresponding flavors. More specifically, the at least one flavor being the carob flavor produces a yogurt with a chocolate flavor. The present invention allows dogs to enjoy the chocolate flavor without experiencing any of the harmful or fatal effects of chocolate.

An alternate embodiment of the present invention comprises at least one human-grade nutritional additive in order for the yogurt premix for canines to be beneficial for humans as well as dogs. The health and taste benefits of the at least one human-grade nutritional additive are gear towards human consumption. The at least one human-grade nutritional additive is heterogeneously mixed into the quantity of powdered yogurt. The at least one human-grade nutritional additive is selected from a group consisting of: glucosamine, chondroitin, and a combination thereof. Glucosamine aids in joint development and joint pain. Chondroitin aids in the bone and tissue repair. It is understood the at least one human-grade nutritional additive may be heterogeneously mixed into the quantity of powdered yogurt given that the at least one human-grade nutritional additive is not harmful or fatal for dogs.

The present invention is a premix that is processed by a soft serve machine. Water is added to the yogurt premix for canines in order for yogurt to be produced via the soft serve machine. It is recommended that a wire whip is used to mix the yogurt premix for canines within the soft serve machine. For better quality yogurt, water is mixed with the yogurt premix for canines and rests for ten minutes, then mixed again by the soft serve machine. In the preferred embodiment of the present invention, the ideal amount of air added to the mixture during aeration is approximately 33% to 45% volume. This amount of air added through aeration enhances both the taste and texture of the premix processed through a soft serve machine. The soft serve product that results from the premix being processed through a soft serve machine is soft and fluffy and is therefore more appealing to dogs. Moreover, the present invention has a much less percentage of fat while preserving a creamy and smooth texture. It is recommended yogurt premix for canines is stored at room temperature until mixed with water.

Although the invention has been explained in relation to its preferred embodiment, it is to be understood that many other possible modifications and variations can be made without departing from the spirit and scope of the invention as hereinafter claimed.

What is claimed is:

1. A yogurt premix for canines comprises:
a quantity of powdered yogurt;
a quantity of protein powder;
a quantity of thickening powder;
a quantity of dried apple;
a quantity of powdered banana;
a quantity of coconut oil;
a quantity of molasses; and
the quantity of protein powder, the quantity of thickening powder, the quantity of dried apple, the quantity of powdered banana, the quantity of coconut oil, and the quantity of molasses being heterogeneously mixed into the quantity of powdered yogurt.

2. The yogurt premix for canines as claimed in claim 1, wherein the quantity of powdered yogurt is approximately 29.2 percent by weight (wt %) of the yogurt premix for canines.

3. The yogurt premix for canines as claimed in claim 1, wherein the quantity of powdered yogurt includes active probiotic cultures.

4. The yogurt premix for canines as claimed in claim 1, wherein the quantity of protein powder is approximately 12.5 wt % of the yogurt premix for canines.

5. The yogurt premix for canines as claimed in claim 1, wherein the quantity of protein powder is whey protein.

6. The yogurt premix for canines as claimed in claim 1, wherein the quantity of thickening powder is approximately 6.3 wt % of the yogurt premix for canines.

7. The yogurt premix for canines as claimed in claim 1, wherein the quantity of dried apple is approximately 7.3 wt % of the yogurt premix for canines.

8. The yogurt premix for canines as claimed in claim 1, wherein the quantity of powdered banana is approximately 12.5 wt % of the yogurt premix for canines.

9. The yogurt premix for canines as claimed in claim 1, wherein the quantity of coconut oil is approximately 7.3 wt % of the yogurt premix for canines.

10. The yogurt premix for canines as claimed in claim 1, wherein the quantity of molasses is approximately 8.3 wt % of the yogurt premix for canines.

11. The yogurt premix for canines as claimed in claim 1 comprises:
a quantity of powdered pumpkin; and
the quantity of powdered pumpkin being heterogeneously mixed into the quantity of powdered yogurt.

12. The yogurt premix for canines as claimed in claim 11, wherein the quantity of powdered pumpkin is approximately 7.3 wt % of the yogurt premix for canines.

13. The yogurt premix for canines as claimed in claim 1 comprises:
a quantity of powdered sweet-potato; and
the quantity of powdered sweet-potato being heterogeneously mixed into the quantity of powdered yogurt.

14. The yogurt premix for canines as claimed in claim 13, wherein the quantity of powdered sweet-potato is approximately 7.3 wt % of the yogurt premix for canines.

15. The yogurt premix for canines as claimed in claim 1 comprises:
a quantity of omega fish oil; and
the quantity of omega fish oil being heterogeneously mixed into the quantity of powdered yogurt.

16. The yogurt premix for canines as claimed in claim 15, wherein the quantity of omega fish oil is approximately 2.1 wt % of the yogurt premix for canines.

17. The yogurt premix for canines as claimed in claim 1 comprises:
at least one flavor additive; and
the at least one flavor additive being heterogeneously mixed into the quantity of powdered yogurt.

18. The yogurt premix for canines as claimed in claim 17, wherein the at least one flavor additive is selected from a group consisting of: a peanut-butter flavor, a carob flavor, a pumpkin flavor, a cinnamon flavor, a sweet-potato flavor, an apple flavor, a coconut flavor, and a combination thereof.

19. The yogurt premix for canines as claimed in claim 1 comprises:
at least one human-grade nutritional additive; and
the at least one human-grade nutritional additive being heterogeneously mixed into the quantity of powdered yogurt.

20. The yogurt premix for canines as claimed in claim 19, wherein the at least one human-grade nutritional additive is selected from a group consisting of: glucosamine, chondroitin, and a combination thereof.
